(19) United States
(12) Patent Application Publication
(10) Pub. No.: US 2011/0020173 A1
(43) Pub. Date: Jan. 27, 2011

(54) Laser Disinfection Apparatus with Spectroscopic Sensor

(75) Inventors: Brian Pryor, Newark, DE (US); Sean Xiaolu Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 12/843,117

(22) Filed: Jul. 26, 2010

Related U.S. Application Data
(60) Provisional application No. 61/228,653, filed on Jul. 27, 2009.

(51) Int. Cl. A61L 2/24; A61B 18/20

(52) U.S. Cl. 422/3; 606/12

(57) ABSTRACT

A laser disinfection apparatus with a spectroscopic sensor for performing real-time monitoring of the apparatus's effectiveness. The spectroscopic sensor measures optical spectra of the biological tissue and obtains the concentration of the bacterial/fungal cells by tracking the intensity variation of a fingerprint region of the optical spectra. The acquired bacterial concentration information is used to evaluate the effectiveness of the laser treatment as well as to provide feed-back control of the laser parameters to obtain the optimum disinfection result.

### REFERENCE TO RELATED APPLICATION

This application claims an invention which was disclosed in Provisional Patent Application No. 61/228,653, filed Jul. 27, 2009, entitled “LASER DISINFECTION APPARATUS WITH SPECTROSCOPIC SENSOR”. The benefit under 35 USC §119(e) of the above mentioned United States provisional applications is hereby claimed, and the aforementioned application is hereby incorporated herein by reference.

### FIELD OF THE INVENTION

This invention generally relates to a laser disinfection apparatus, and more specifically to a laser disinfection apparatus with a spectroscopic sensor.

### BACKGROUND

Laser therapy was demonstrated to be an effective method for killing bacterial/fungal cells and had been successfully applied for the treatment of periodontal diseases, acnes, onychomycosis, etc. The disinfection function of laser light is fulfilled either through thermo-damage to the bacteria (where the bacteria are killed by a temperature rise induced by the laser energy) or through certain kind of photo damage (where the laser energy is believed to be absorbed by bacterial chromophores to produce bactericidal singlet oxygen).

The effectiveness of a laser disinfection apparatus is affected by a variety of parameters, such as laser wavelength, power density, mode of operation (continuous mode vs. pulsed mode), and the optical properties (e.g. absorption coefficient, scattering coefficient, refractive index) of the infected biological tissue. However, none of the current available laser disinfection apparatus provides means to evaluate its own effectiveness. As a result, the operator has to control the laser parameters based on his/her past experience, which may not yield the optimum disinfection result.

### SUMMARY OF THE INVENTION

It is thus the overall goal of the present invention to solve the above-mentioned problem and provide a laser disinfection apparatus with a spectroscopic sensor for performing real-time monitoring of the apparatus's effectiveness. The spectroscopic sensor measures optical spectra of the infected biological tissue and obtains the concentration of the bacterial/fungal cells by tracking the intensity variation of a fingerprint region of the optical spectra. The acquired bacterial concentration information can be used to evaluate the effectiveness of the laser treatment as well as to provide feed-back control of the laser parameters to obtain the optimum disinfection result.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the laser disinfection apparatus with spectroscopic sensor.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to laser disinfection apparatus with spectroscopic sensor. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

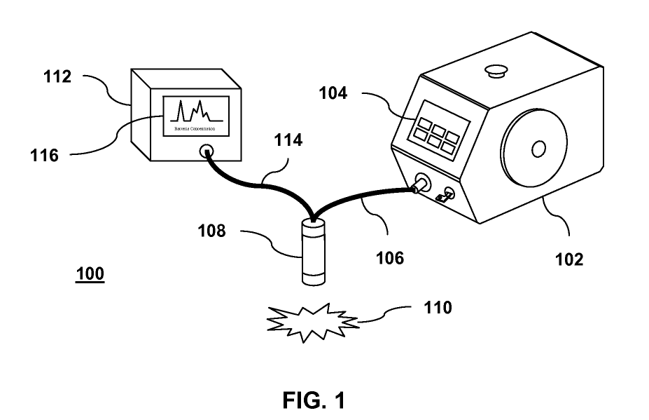

One exemplary embodiment of the present invention is shown in FIG. 1. The laser disinfection apparatus 100 comprises a laser unit 102 and a spectroscopic sensor unit 112. The laser unit 102 comprises a laser light source (preferably consisting of one or more laser diodes) (not shown) for producing laser light. The laser light is coupled into a light guide 106 and delivered through a hand piece 108 to the infected biological tissue 110. By properly selecting the laser wavelength, the laser light can be effectively absorbed by certain bacterial chromophores to produce reactive chemical species such as singlet oxygen, which destroy the infection bacteria/fungus. It is also possible to use the laser light to excite a vibrational state of the microorganisms to diminish their activity. As another approach, the laser wavelength can be selected to match with the absorption band of water content of the bacterial/fungal cells. Thus the absorbed laser light induces a temperature rise hence causes thermal damage to the microorganisms. The laser light source may consist of multiple laser diodes with different output wavelengths (ultraviolet, visible, infrared, etc.). Each laser wavelength is selected to target a specific kind of bacteria/fungus. The multiple laser wavelengths may be applied simultaneously to achieve the optimum disinfection result. The laser unit 102 further comprises a touch-screen display/control unit 104, which is used to display and control the current operation parameters of the laser light source (e.g. laser wavelength, average power level, time of duration, pulse energy, peak power, duty cycle, repetition rate).

The spectroscopic sensor unit 112 utilizes infrared (IR) spectroscopy, Raman spectroscopy, or fluorescence spectroscopy techniques to monitor the variation of bacteria/fungus concentration before/during/after laser treatment. The spectroscopic sensor unit 112 may comprise a light source, such as a lamp, a laser or a light emitting diode (LED) (not shown), to produce optical radiation, which is then delivered through a light guide 114 and the hand piece 108 to illuminate the infected biological tissue 110. An absorption/reflection, fluorescence, or Raman spectrum of the biological tissue 110 is obtained by measuring the spectral intensity distribution of the transmitted/reflected, fluorescence, or Raman scattering optical signal from the tissue and displayed on a display unit 116. By analyzing certain finger print regions (e.g. amide region at around 1500-1600 cm⁻¹, fatty acid region at around 2800-3000 cm⁻¹) of the obtained optical spectrum, the bacteria/fungus that infect the biological tissue can be identified and their concentration can be estimated. The incorporation of the spectroscopic sensor unit 112 provides three advantages. First, the operator can select the appropriate laser parameters, such as laser wavelength, power level, etc. according to the types of bacteria/fungus identified. Second, the variation of bacteria/fungus concentration before/after laser treatment can be used to evaluate the effectiveness of the laser disinfection apparatus. Third, the acquired bacteria/fungus concentration information can be used to provide feed-back control of the laser parameters to achieve the optimum disinfection result. Such parameters include but are not limited to laser wavelength, average power level, time of duration, pulse energy, peak power, duty cycle, repetition rate, etc.

In a slight variation of the embodiment, the spectroscopic sensor unit 112 can be directly integrated into the laser unit 102 instead of being used as a stand-alone device. The laser light source may be replaced with other kind of light sources such as light emitting diodes (LEDs), super-luminescence diodes (SLDs), or lamp light sources.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A laser disinfection apparatus for killing bacterial/fungal microorganisms of infected biological tissue, the laser disinfection apparatus comprising:
at least one laser light source for producing laser light to cause photo damage or thermal damage to the bacterial/fungal microorganisms; and
at least one spectroscopic sensor for measuring an optical spectrum of the bacterial/fungal microorganisms and obtaining identification and concentration information of the bacterial/fungal microorganisms from said optical spectrum;
wherein said identification and concentration information is utilized to control a set of parameters and evaluate the effectiveness of said at least one laser light source.

2. The laser disinfection apparatus of claim 1, wherein the laser light source comprises at least one laser diode.

3. The laser disinfection apparatus of claim 1, wherein the laser light source comprises at least two laser diodes with different output wavelengths.

4. The laser disinfection apparatus of claim 1, wherein the output wavelength of the laser light source falls in a range from ultraviolet to infrared.

5. The laser disinfection apparatus of claim 1, wherein the optical spectrum comprises at least one of absorption/reflection spectrum, fluorescence spectrum, or Raman spectrum.

6. The laser disinfection apparatus of claim 1, wherein the set of parameters comprise at least one of laser wavelength, power level, time of duration, pulse energy, peak power, duty cycle, or repetition rate.

7. A method for killing bacterial/fungal microorganisms of infected biological tissue, the method comprising the steps of:
providing at least one laser light source for producing laser light to cause photo damage or thermal damage to the bacterial/fungal microorganisms;
providing at least one spectroscopic sensor for measuring an optical spectrum of the bacterial/fungal microorganisms and obtaining identification and concentration information of the bacterial/fungal microorganisms from said optical spectrum; and
utilizing said identification and concentration information to control a set of parameters and evaluate the effectiveness of said at least one laser light source.
